(19) United States
(12) Patent Application Publication
(10) Pub. No.: US 2016/0151088 A1
(43) Pub. Date: Jun. 2, 2016

(54) SAFETY PROTECTIVE NEEDLE COVER FOR BUTTERFLY NEEDLE ASSEMBLY

(71) Applicant: Perfect Medical Industry Co., Ltd, CHANGHUA (TW)

(72) Inventors: KUO-CHI HSU, CHANGHUA (TW); CHENG- KUO CHUANG, CHANGHUA (TW); MIN-SUNG LAI, CHANGHUA (TW)

(21) Appl. No.: 14/558,457

(22) Filed: Dec. 2, 2014

(51) Int. Cl. A61B 17/34; A61M 5/158

(52) U.S. Cl. CPC A61B 17/3494; A61M 5/158; A61M 2005/1581; A61M 2005/1587

(57) ABSTRACT

A safety protective needle cover for butterfly needle assembly is disclosed. After the covering member is pivoted toward the base, the base may be covered by an accommodating space defined by the backs of the two pivoting walls and the concave cover such that the covering member and the base are parallel with each other. The needle will not be shifted due to the shake of the patient or being hooked by dress. After the covering member is pivoted toward the vertical portion of the L-shaped needle, the vertical portion of the L-shaped needle may be covered by the concave cover to prevent nursing staff or users from being stuck.

### FIELD OF THE INVENTION

The present invention relates to a butterfly needle, and more particularly to a safety protective needle cover for butterfly needle assembly.

### BACKGROUND OF THE INVENTION

Accidental needle sticks can occur in several ways. For example, a sudden movement by a patient can cause a healthcare worker to lose control of the needle, resulting in injury. Additionally, injuries can result when contaminated, unprotected needles are left unattended or disposed of improperly. Moreover, attempts to manually recap a needle after a medical procedure can also result in injury. In addition to accidental needle sticks, unnecessary exposure to bloodborne pathogens can result when a healthcare worker mistakenly re-uses a contaminated needle on a patient.

Accidental needle sticks and the inadvertent reuse of a contaminated needle have the potential to expose patients and healthcare workers to life-threatening viruses that include hepatitis and HIV. Because of this potential exposure, healthcare providers are obligated to conduct extensive testing of exposed individuals. Additional follow-up testing for HIV is typically prescribed approximately six months after the exposure. It is to be appreciated that the costs associated with the testing, lab work, the workers lost time, and the associated tracking and administrative costs, can be considerable.

One type of needle device that can cause accidental needle sticks is the butterfly needle. The butterfly needle is typically used when it is required to introduce a fluid into or withdraw a fluid from a patient over a relatively long period of time. For example, the butterfly needle can be connected to a syringe, an IV set or a blood collection holder. One advantageous feature of a butterfly needle is that it contains wings that can be folded flat and taped to the patient's skin to stabilize the device. This is especially important when the needle must remain in place for a relatively long period. Typically, the wings of a butterfly needle are initially juxtaposed to allow the healthcare worker to hold the device during insertion of the needle into the patient. After needle insertion, the wings are folded flat against the patient's skin and taped. To remove the butterfly needle, the tape is pulled up and the wings are folded together (i.e. juxtaposed). With the wings together, the needle can be easily withdrawn from the patient.

### SUMMARY OF THE INVENTION

An objective of this invention is providing a safety protective needle cover for butterfly needle assembly. After the covering member is pivoted toward the base, the base may be covered by an accommodating space defined by the backs of the two pivoting walls and the concave cover such that the covering member and the base are parallel with each other. The needle will not be shifted due to the shake of the patient or being hooked by dress.

Additionally, after the covering member is pivoted toward the vertical portion of the L-shaped needle, the vertical portion of the L-shaped needle may be covered by the concave cover to prevent nursing staff or users from being stuck.

To achieve above objectives, a safety protective needle cover for butterfly needle assembly is provided. The safety protective needle cover for butterfly needle assembly may comprise a base, having a receiving groove disposed at a bottom thereof; two wings, respectively extending from two sides of the base outwardly; an L-shaped needle, having a horizontal portion and a vertical portion connected with each other, the horizontal portion is inserted into the receiving groove of the base, and the vertical portion is exposed outside the receiving groove and arranged downwardly; and a covering member, having two pivoting walls and a concave cover, the two pivoting walls are respectively and pivotably connected with two sides of one end of the base adjacent to the vertical portion, and the concave cover is axially disposed between the two pivoting walls and parallel to an axial direction of the two pivoting walls.

In one embodiment, two first pressing members are respectively extended relatively from two inner walls of the concave cover, and the vertical portion of the L-shaped needle is pressed into the concave cover through the two first pressing members and then the two first pressing members are against the vertical portion of the L-shaped needle to be fastened in the concave cover.

In another embodiment, two second pressing members are respectively and alternatively extended from the two inner walls of the concave cover, the two second pressing members are spaced apart from the first pressing members, and the vertical portion of the L-shaped needle is pressed into the concave cover through the two first pressing members and the two second pressing members and then the two first pressing members and the two second pressing members are simultaneously against the vertical portion of the L-shaped needle to be fastened in the concave cover.

Further features and advantages of the present invention will become apparent to those of skill in the art in view of the detailed description of preferred embodiments which follows, when considered together with the attached drawings and claims.

### BRIEF DESCRIPTION OF THE DRAWINGS

All the objects, advantages, and novel features of the invention will become more apparent from the following detailed descriptions when taken in conjunction with the accompanying drawings.

FIG. 1 is an exploded view of a safety protective needle cover for butterfly needle assembly according to this present invention.

FIG. 2 is an outside view of the safety protective needle cover for butterfly needle assembly according to this present invention.

FIG. 3 is an another outside view of FIG. 2.

FIG. 4 is a cross-sectional view of the safety protective needle cover for butterfly needle assembly according to this present invention before a covering member is pivoted toward a vertical portion of the L-shaped needle.

FIG. 5 is a cross-sectional view of FIG. 4 while the vertical portion of the L-shaped needle is covered by the covering member.

FIG. 6 is a cross-sectional view of the safety protective needle cover for butterfly needle assembly according to this present invention before a covering member is pivoted toward a base.

FIG. 7 is a cross-sectional view of FIG. 6 while the base is covered by the covering member.

### DESCRIPTION OF THE PREFERRED EMBODIMENT

Referring now to the drawings where like characteristics and features among the various figures are denoted by like reference characters.

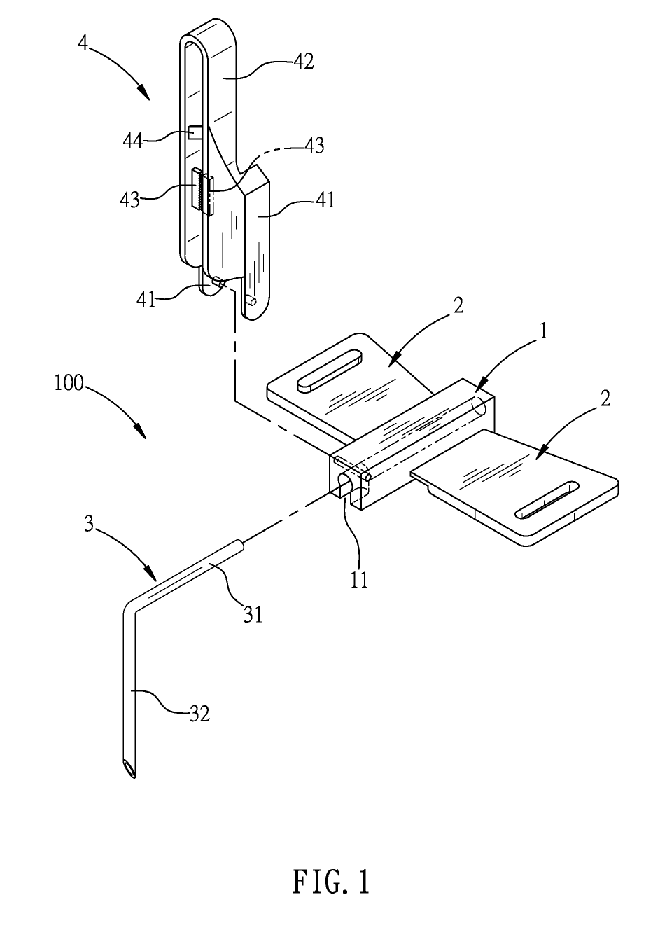

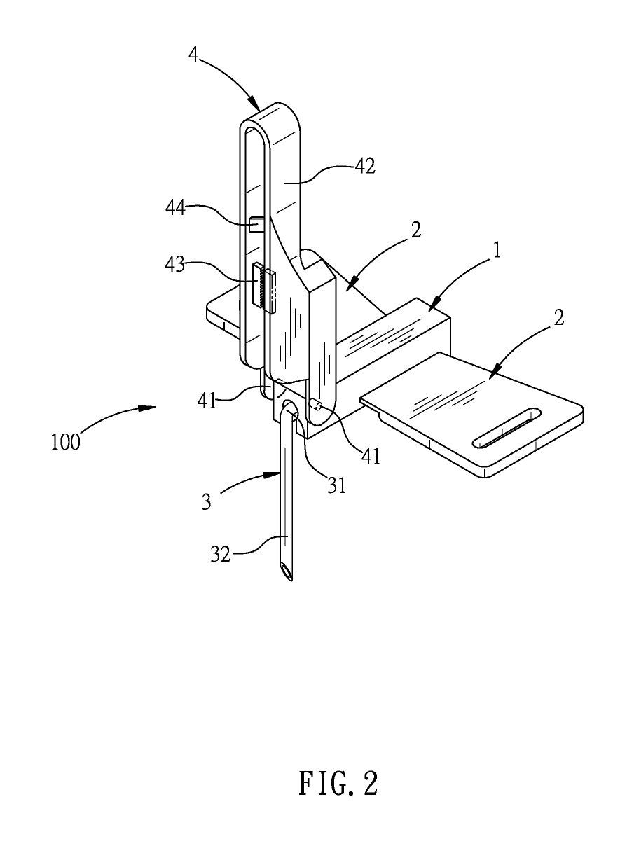

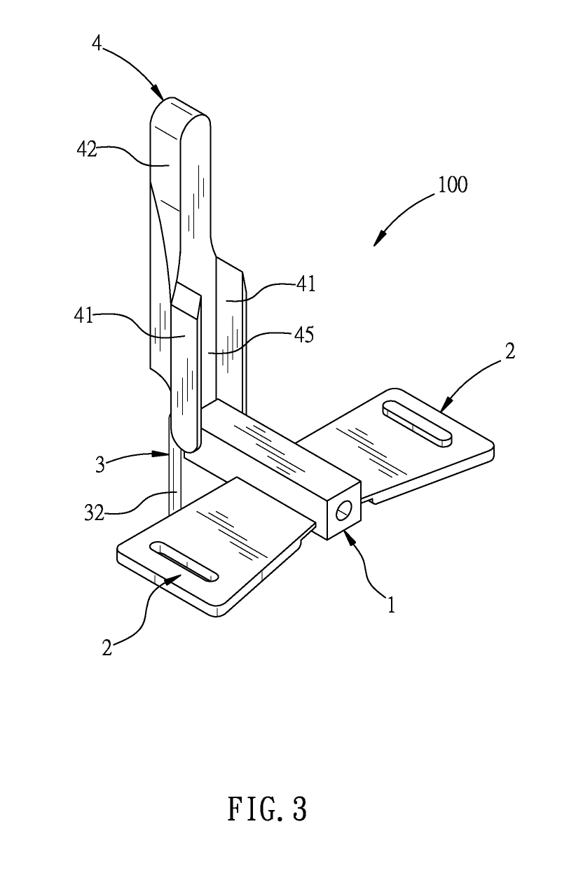

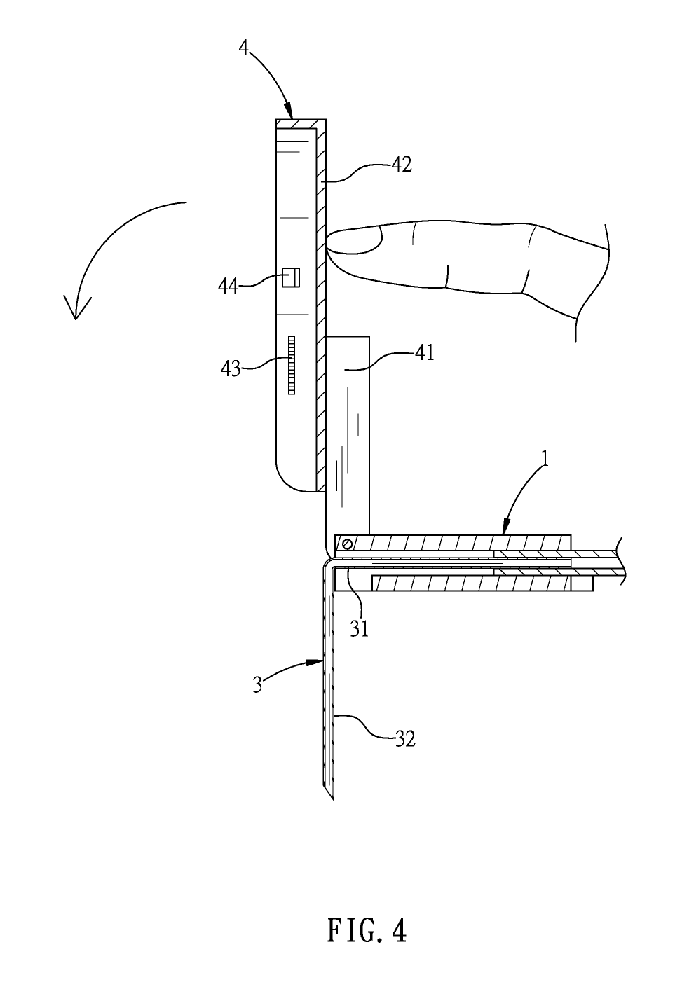

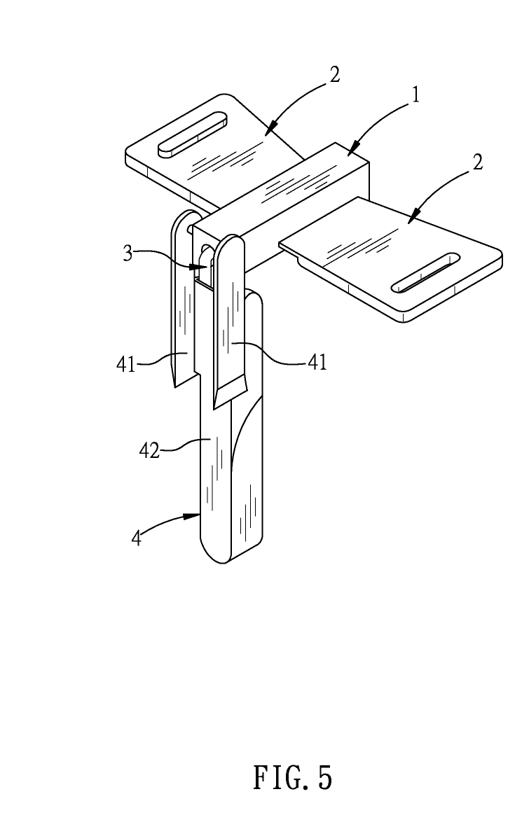

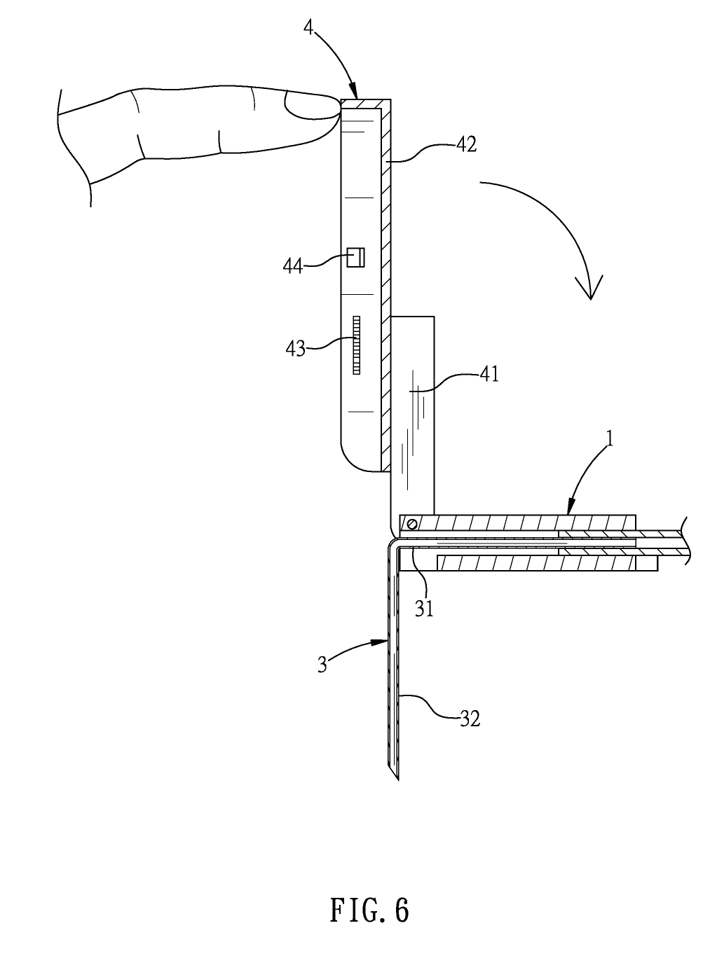

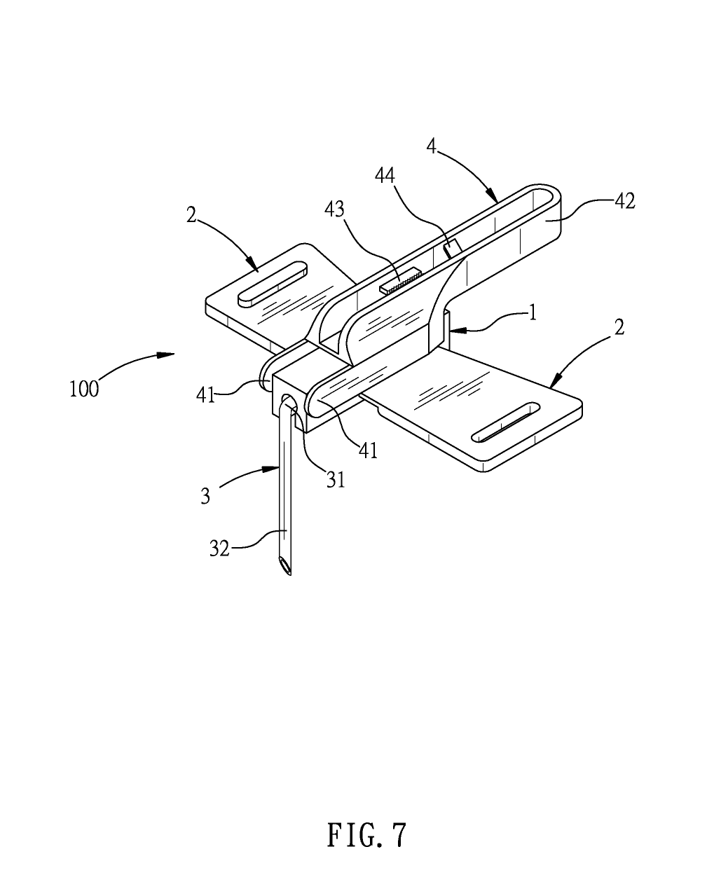

FIG. 1 is an exploded view of a safety protective needle cover for butterfly needle assembly according to this present invention. FIG. 2 is an outside view of the safety protective needle cover for butterfly needle assembly according to this present invention. FIG. 3 is an another outside view of FIG. 2. FIG. 4 is a cross-sectional view of the safety protective needle cover for butterfly needle assembly according to this present invention before a covering member is pivoted toward a vertical portion of the L-shaped needle. FIG. 5 is a cross-sectional view of FIG. 4 while the vertical portion of the L-shaped needle is covered by the covering member. FIG. 6 is a cross-sectional view of the safety protective needle cover for butterfly needle assembly according to this present invention before a covering member is pivoted toward a base. FIG. 7 is a cross-sectional view of FIG. 6 while the base is covered by the covering member.

Please refer to FIGS. 1 to 7, the safety protective needle cover 100 for butterfly needle assembly may comprise a base 1, two wings 2, an L-shaped needle 3, and a covering member 4.

The base 1 may have a receiving groove 11 disposed at a bottom thereof.

Two wings 2 may be respectively extending from two sides of the base 1 outwardly.

The L-shaped needle 3 may have a horizontal portion 31 and a vertical portion 32 connected with each other. The horizontal portion 31 may be inserted into the receiving groove 11 of the base 1 and the vertical portion 32 may be exposed outside the receiving groove 11 and arranged downwardly.

The covering member 4 may have two pivoting walls 41 and a concave cover 42. The two pivoting walls 41 may be respectively and pivotably connected with two sides of one end of the base 1 adjacent to the vertical portion 31 of the L-shaped needle 3, and the concave cover 42 may be axially disposed between the two pivoting walls 41 and parallel to an axial direction of the two pivoting walls 41.

After the covering member 4 is pivoted toward the base 1 (shown as in FIGS. 6 and 7), the base 1 may be covered by an accommodating space defined by the backs of the two pivoting walls 41 and the concave cover 42 such that the covering member 4 and the base 1 are parallel with each other. The needle 3 will not be shifted due to the shake of the patient or being hooked by dress.

After the covering member 4 is pivoted toward the vertical portion 32 of the L-shaped needle 3 (shown as in FIGS. 4 and 5), the vertical portion 32 of the L-shaped needle 3 may be covered by the concave cover 42 to prevent nursing staff or users from being stuck.

In addition, two first pressing members 43 may be respectively extended relatively from two inner walls of the concave cover 42. The vertical portion 32 of the L-shaped needle 3 may be pressed into the concave cover 42 through the two first pressing members 43. And then the two first pressing members 43 may be against the vertical portion 32 of the L-shaped needle 3 to be fastened in the concave cover 42.

Furthermore, two second pressing members 44 may be respectively and alternatively extended from the two inner walls of the concave cover 42. The two second pressing members 44 may be spaced apart from the first pressing members 43. And the vertical portion 32 of the L-shaped needle 3 may be pressed into the concave cover 42 through the two first pressing members 43 and the two second pressing members 44. And then the two first pressing members 43 and the two second pressing members 44 may be simultaneously against the vertical portion 32 of the L-shaped needle 3 to be fastened in the concave cover 42.

The two first pressing members 43 and the second pressing members 44 may ensure that the vertical portion 32 of the L-shaped needle 3 is covered and fastened in the concave cover 42 to prevent nursing staff or users from being stuck.

What is claimed is:

1. A safety protective needle cover for butterfly needle assembly, comprising:
a base, having a receiving groove disposed at a bottom thereof;
two wings, respectively extending from two sides of the base outwardly;
an L-shaped needle, having a horizontal portion and a vertical portion connected with each other, the horizontal portion is inserted into the receiving groove of the base, and the vertical portion is exposed outside the receiving groove and arranged downwardly; and
a covering member, having two pivoting walls and a concave cover, the two pivoting walls are respectively and pivotably connected with two sides of one end of the base adjacent to the vertical portion, and the concave cover is axially disposed between the two pivoting walls and parallel to an axial direction of the two pivoting walls;
wherein after the covering member is pivoted toward the base, the base is covered by an accommodating space defined by the backs of the two pivoting walls and the concave cover such that the covering member and the base are parallel with each other; and after the covering member is pivoted toward the vertical portion of the L-shaped needle, the vertical portion of the L-shaped needle is covered by the concave cover.

2. The safety protective needle cover for butterfly needle assembly as claimed in claim 1, wherein two first pressing members are respectively extended relatively from two inner walls of the concave cover, and the vertical portion of the L-shaped needle is pressed into the concave cover through the two first pressing members and then the two first pressing members are against the vertical portion of the L-shaped needle to be fastened in the concave cover.

3. The safety protective needle cover for butterfly needle assembly as claimed in claim 2, wherein two second pressing members are respectively and alternatively extended from the two inner walls of the concave cover, the two second pressing members are spaced apart from the first pressing members, and the vertical portion of the L-shaped needle is pressed into the concave cover through the two first pressing members and the two second pressing members and then the two first pressing members and the two second pressing members are simultaneously against the vertical portion of the L-shaped needle to be fastened in the concave cover.
